(19) United States
(12) Patent Application Publication
(10) Pub. No.: US 2013/0273830 A1
(43) Pub. Date: Oct. 17, 2013

(54) Method and apparatus for molecular evaporation of fragrance substances/active substances and their dissemination

(71) Applicants: Georg Ortner, (US); LINDE AG, Munchen (DE)

(72) Inventor: Georg Ortner, Koln (DE)

(73) Assignees: Linde AG, Munchen (DE); Georg Ortner, Koln (DE)

(21) Appl. No.: 13/860,630

(22) Filed: Apr. 11, 2013

(30) Foreign Application Priority Data
Apr. 12, 2012 (DE) 102012007167.1

(51) Int. Cl. A61L 9/03

(52) U.S. Cl. CPC A61L 9/03
USPC 454/337

(57) ABSTRACT

In the case of a method for molecular evaporation of fragrance substances and/or active substances (FA substances) and their dissemination by way of air streams in the surrounding air, in closed rooms, in ducts of ventilation and air conditioning systems, or the like, wherein FA substance, before its dissemination, is applied, in liquid form, in targeted manner, to a strip-shaped FA-substance carrier (6), it is provided
that the strip-shaped FA-substance carrier (6) is moved to circulate at least partly within a dissemination zone for FA substance, and
that the FA substance to be evaporated is heated, directly or indirectly, to a temperature that promotes molecular evaporation, during its circulation on the FA-substance carrier (6).

The invention relates to a method and an apparatus in accordance with the preambles of the independent claims.

In an International Patent Application having the publication number WO 2011/054529 A1, a method and an apparatus for generation of a fragrance/air stream for fragrance diffusion in rooms, premises, or the like are described, whereby the fragrance substance is sprayed onto a strip-shaped fragrance substance carrier, in targeted manner, which carrier is moved through an evaporation shaft for the purpose of evaporation of the fragrance substance, through which shaft fresh air flows. After it exits from the evaporation shaft, the used section of the fragrance substance carrier is disposed of as waste. The particular advantage, in this connection, is that mixing of the fragrance substance into the fresh air stream can take place without undesirable deposits of liquid fragrance substances on parts of the apparatus, so that no contamination or clogging of the apparatus occurs. Instead, the fragrance substance is picked up by the fresh air that flows in the evaporation shaft, in molecular form, after prior evaporation of the fragrance substance applied to the fragrance substance carrier in liquid form. In this connection, the evaporation process can be further improved by supplying heated fresh air, i.e. in this way, extensive evaporation is achieved within a shorter time.

In this connection, fragrance diffusion in rooms can take place automatically, to a great extent, whereby the amount of fragrance substance evaporated per time unit can be changed in accordance with program defaults, by means of an electronic control device.

In contrast, the present invention is based on the task of creating a method and an apparatus in which the evaporation process takes place particularly effectively and in cost-saving manner, whereby the process can be controlled automatically according to program defaults, and whereby the dissemination of fragrance substances and/or active substances takes place in particularly effective manner, regardless of whether this occurs as part of free air flow, for example under convection or by way of air conditioning or ventilation systems, and even at relatively high flow velocities of the inflowing fresh air.

This task is accomplished, according to the invention, according to a method having the characteristics of the independent claims.

For this purpose, it is provided, in the case of a method for molecular evaporation of fragrance substances and/or active substances (FA substances) and their dissemination by way of air streams in the surrounding air, in closed rooms, in ducts of ventilation and air conditioning systems, or the like, whereby FA substance, before its dissemination, is applied, in liquid form, to a strip-shaped FA-substance carrier, and whereby the FA-substance carrier charged with FA substance has an air stream flowing against it transverse to its longitudinal axis,

that the strip-shaped FA-substance carrier is moved to circulate at least partly within a dissemination zone for FA substance, and

that the FA substance to be evaporated is heated, directly or indirectly, to a temperature that promotes molecular evaporation, during its circulation on the FA-substance carrier.

With this method, it is possible to generate an air stream charged with FA substance, of any desired volume, continuously and at always the same quality, at a quantity of the molecularly evaporated FA substance that can be metered precisely, i.e. at a predetermined FA-substance intensity. Under controlled, uniform evaporation, an air stream enriched with FA substance according to a predetermined program, for example, at a uniform quality, is obtained, independent of whether the inflow to the circulating FA-substance carrier takes place using suction flow or blowing flow of the fresh air to be treated. The evaporation surface area can be adapted as a function of the flow volume of the fresh air, from a few mm² for smaller rooms to multiple m²; for example for continuous fragrance diffusion in landscapes or for treatment of landscapes with active substances.

Thrifty consumption of FA substances is guaranteed in that the FA-substance carrier is heated, directly or indirectly, to a temperature that promotes evaporation. In this connection, the heat can be transferred more or less directly to the FA-substance carrier. Indirect heating by means of a partial air stream in the immediate vicinity of the FA-substance carrier is also possible.

A circulating movement of the strip-shaped FA-substance carrier on rollers, within the dissemination zone, is particularly advantageous. Important application cases are, in this connection, the introduction of FA substance into door air curtains, for example of hotel entrances, entrances to stores, exhibition centers, or the like. In this connection, uniform fragrance dissemination over the entire door width is particularly important; this can advantageously be implemented, in reliable manner, by means of a strip-shaped FA-substance carrier that circulates between deflection rollers.

Fragrance diffusion in rooms, by means of FA substances, also plays an increasingly greater role, for example in the business spaces of manufacturers of brand name goods, fashion houses, or other spaces for advertising events, exhibitions, etc.

In these application cases, the dissemination zone extends, for example, over the entire width of a room entrance, an entry door or the like. According to the invention, it should be aimed at, in this connection, that in order to achieve a uniform FA-substance impact on the air stream to be affected, the circulation length of the FA-fragrance carrier that is moved on rollers is adapted to the width of the flow passage, to the greatest possible extent.

To control the FA-substance intensity, it is provided, according to the invention, that the FA-substance carrier can be moved at a variably adjustable speed or that the amount of the FA substance applied to the FA-substance carrier in liquid form is adjustable. In this sense, the possibility exists, according to the invention, of constantly supplying the FA-substance carrier with fresh FA substance, by means of corresponding programming of a metering apparatus, whereby time intervals and metered substance amounts are variably adjustable.

To regulate the evaporation temperature, it is provided, in an embodiment of the method according to the invention, that a suitable temperature in the range between 40° and 100° C. can be selected by means of direct or indirect heating of the FA-substance carrier. Preferably, an evaporation temperature of approx. 60°-80° C. is used. In the case of FA substances that contain crystalline substances, significantly higher temperatures are also possible.

A heating effect extending spatially over the entire width of the air stream to be treated, if possible, is advantageous in the interests of complete evaporation of the applied FA substance, i.e. not only the spatial expanse of the circulating FA-substance carrier but also that of the heating apparatus assigned to it is important for the effectiveness of the method according to the invention.

An apparatus according to the invention, for implementing the method described above, for molecular evaporation and dissemination of fragrance substances/active substances by way of air streams in the surrounding air, in closed rooms, in ducts of ventilation and air conditioning systems, or the like, comprises a metering apparatus for application of FA substance, in liquid form, to a strip-shaped FA-substance carrier, which moves at least partly within a dissemination zone, and, in this connection, has an air stream flowing against it transverse to its longitudinal axis, and whereby the metering apparatus is disposed outside of the dissemination zone.

The apparatus according to the invention particularly comprises a strip-shaped FA-substance carrier, which can be moved on a support profile to circulate on rollers, as well as a heating apparatus that essentially extends along the movement path of the FA-substance carrier.

Ultimately, complete evaporation of the applied FA substance and its uniform dissemination over the entire width of a dissemination zone can be achieved in this way, without the air stream to be treated in this connection being heated as a whole. Instead, it is sufficient to heat the FA-substance carrier, i.e. to bring it to a temperature that promotes evaporation.

This can take place, according to another embodiment, in particularly energy-saving manner, in that the strip-shaped FA-substance carrier that circulates on rollers as well as the related heating apparatus are disposed in encapsulated manner, at least within the dissemination zone, except for incoming and outgoing ventilation openings.

A heating apparatus in the form of an elongated heating rod, which is disposed within the capsule, between an upper and a lower run of the FA-substance carrier that circulates around between two deflection rollers, is particularly advantageously suitable for this purpose.

In this connection, the heating rod radiates its heat uniformly onto the circulating FA-substance carrier, over its entire length, so that direct evaporation of the FA substance applied to the FA-substance carrier in liquid form comes about. A small proportion of fresh air that penetrates through the incoming ventilation opening is eddied about in the capsule and ensures evaporation of the FA substance in molecular form, and its dissemination through outgoing ventilation openings out of the capsule, into the air stream that is flowing past on the outside.

A hollow profile that has narrow slit openings on opposite sides, on the one hand for entry of fresh air into the capsule, on the other hand for its exit with the FA-substance molecules that have been taken up, so that these are distributed in the surrounding air stream, is particularly advantageously suitable as a capsule.

It is advantageous if the hollow profile is composed of profile parts that delimit narrow slits between them or have one or more slit-shaped openings.

It is advantageous if the profile parts are accommodated in a U-shaped outer profile that has flow-advantageous profile projections on one or both sides. These projections serve to generate a suction effect on an outside of the hollow profile to which the slit openings are assigned.

In order to save weight, it is practical if the profile parts and the outer profile consist of light metal, for example of aluminum.

A U profile made of heat-insulating material is inserted between the profile parts and the outer profile that surrounds them.

This U profile serves to store heat generated by the heating rod in the interior of the hollow profile, while simultaneously minimizing energy loss. Alternatively, the outer profile itself can also consist of heat-insulating material. The said U profile can then be eliminated.

A suitable FA-substance carrier consists of a metal strip tightly bound of individual threads, with a correspondingly great capillary effect. Such a metal strip, which is formed, for example, in the form of a braided strip from braided metal threads, possesses a long useful lifetime; because of its inherent capillary effect, it accommodates the FA substances applied to it in liquid form without dripping, and allows their problem-free evaporation, promoted by the heat action of the heating rod disposed in the immediate vicinity of the FA-substance carrier.

Before any change in the FA substance, such a metal strip can either be cleaned, or replaced after extended use.

For an FA-substance carrier that circulates between two deflection rollers, and can have a length from 0.2 to several meters, depending on the expanse of the dissemination zone, it is advantageous that one of the deflection rollers is configured as a drive roller, outside of the dissemination zone, and driven by an electric motor coupled to it.

As described above, two essential components of the object of the invention, namely the circulating FA-substance carrier and the related heating apparatus, are accommodated within a hollow profile that serves as encapsulation of these components. During installation, the apparatus according to the invention is advantageously fastened onto a ventilation duct, in such a manner that its hollow profile projects freely through a wall opening of the ventilation duct, into its interior, in the manner of a lance, and is fastened onto the wall of the ventilation duct only with one end. Further components of the apparatus according to the invention are situated on the outside of the wall, such as, for example, an outer, driven deflection roller with drive motor, the metering apparatus, the electronic control, as well as an FA-substance supply container.

The installation position of the hollow profile can be selected as desired, between a horizontal and a vertical position, preferably in a plane transverse to the longitudinal direction of the air stream guided in the ventilation duct, in other words usually transverse to the ventilation duct.

Aside from ventilation ducts of ventilation and air conditioning systems, supply ducts of door air curtains are also possible as a preferred area of application. In this connection, the apparatus according to the invention can also be disposed directly ahead of the intake region of the unit that generates the door air curtain, for example in a separate housing made of perforated sheet metal.

In this sense, in a further embodiment of the present invention, it is provided that in the case of an apparatus for generating a door air curtain, the apparatus according to the invention is disposed entirely or partly outside of or within the flow duct for the incoming air, and that this air flows against it transverse to its longitudinal axis.

In the following, an exemplary embodiment of the apparatus according to the invention will be described using the drawing. This shows:

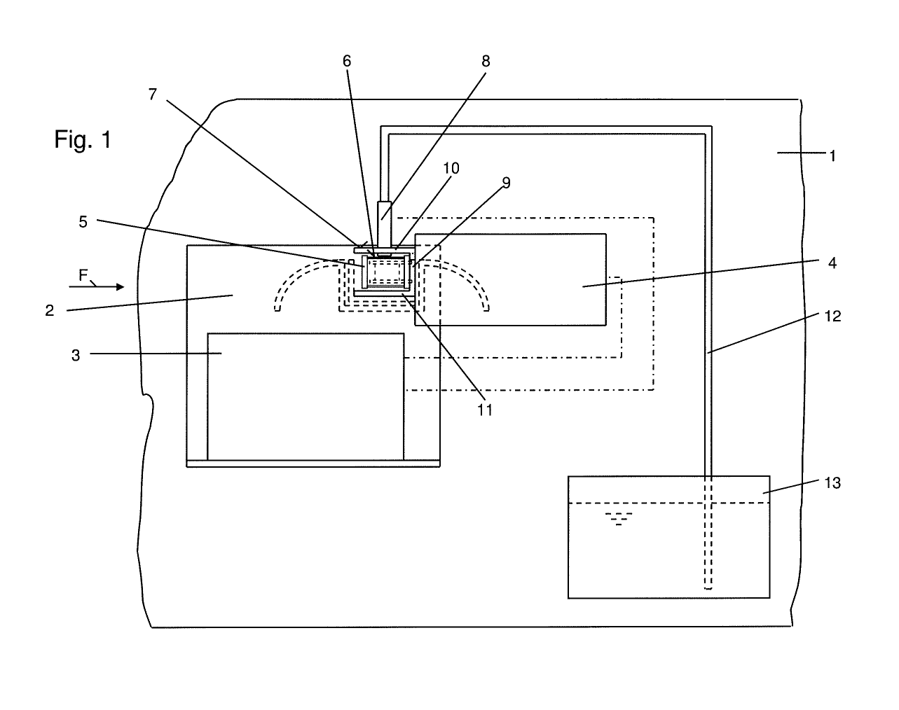

FIG. 1 a schematic system view of the evaporation apparatus,

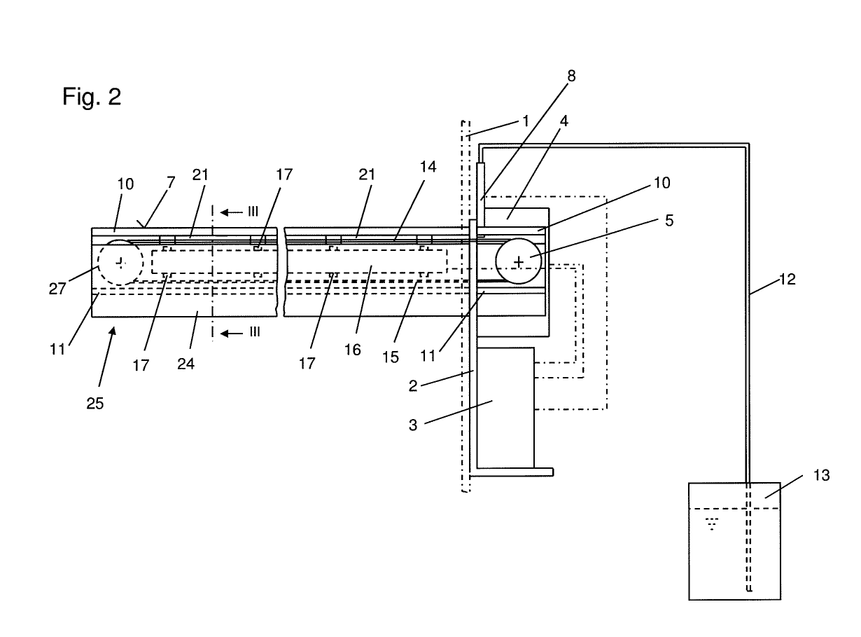

FIG. 2 a longitudinal section through the evaporation apparatus in encapsulated form, and

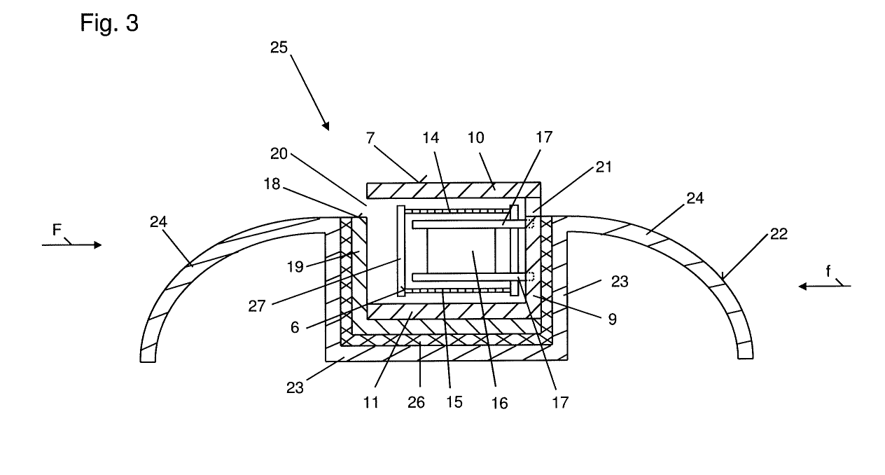

FIG. 3 a cross-section through the evaporation apparatus according to III-III of FIG. 2.

FIG. 1 shows a schematic view of the evaporation apparatus, with a view toward the outside of a side wall 1 of a ventilation duct, onto which an installation plate 2 with the parts of the evaporation apparatus installed on it is fastened. These parts include an electronic control unit 3, an electric motor 4, an FA-substance nozzle 8 of a metering apparatus, which is connected with a supply container 13, in which FA substance is situated in liquid form, by way of an FA-substance supply line 12, which can act as a pressure line or a suction line.

The motor 4 serves to drive a driven deflection roller 5 for circulation of a strip-shaped FA-substance carrier 6, to which FA substance in liquid form is applied by means of the nozzle 8, which roller is disposed outside of the ventilation duct. Not only the nozzle 8 but also the electric motor 4 are fastened onto an end of a U profile 7 situated outside of the side wall 1, in the interior of which profile the circulating FA-substance carrier 6 is accommodated. The electric motor 4 and the nozzle 8 are firmly connected to the U profile 7. The drive shaft between the electric motor 4 and the driven deflection roller 5 is not shown in the drawing; it extends from the electric motor 4 through the crosspiece 9 of the U profile 7, where it is connected with the deflection roller 5 in torque-proof manner. The two shanks 10, 11 of the U profile 7 point into the interior of the ventilation duct, opposite the inflow direction of the air, according to the arrow F, so that a slight proportion of flow on the open side of the U profile 7 can get into its interior, and can flow around the FA-substance carrier 6 there, from all sides.

The nozzle 8 sits in the upper shank 10 of the U profile 7, which is preferably configured as an AL profile.

The control unit 3 is connected with the electric motor 4, the nozzle 8, and with a heating rod 16 (FIG. 2) by way of control lines indicated with dot-dash lines, so that its automatic activation, by way of a computer program controlled by means of corresponding sensors, which monitor metering, among other things, is possible.

In FIG. 2, the part of the evaporation apparatus disposed within the ventilation duct is also shown in the drawing. The apparatus parts situated outside of the side wall 1 of the ventilation duct are provided with the same reference symbols as in FIG. 1.

The U profile 7 is part of an encapsulation of the hollow-profile carrier 25 of the evaporation apparatus that projects into the interior of the ventilation duct, in rod shape. This hollow-profile carrier 25 is shown in an enlarged representation in FIG. 3, which corresponds to a section in the plane III-III of FIG. 2. In FIG. 2, the sectional view of the hollow-profile carrier 25 is shown with broken lines; this is clearly enlarged in FIG. 3. In the interior of the U profile 7, aside from the strip-shaped FA-substance carrier 6 with an upper run 14 and lower run 15, there is a heating rod 16 that is fastened onto the crosspiece 9 of the U profile 7, between upper and lower holding pins 17.

The U profile 7 is mounted on an L profile 18 (FIG. 3), in such a manner that its shanks 19, which are parallel to the crosspiece 9, close the U profile 7 on the open side, to a great extent. Only a narrow gap 20 (FIG. 3) remains between the end of the shank 19 of the L profile 18 and the adjacent end of the upper shank 10 of the U profile 7, through which gap a small proportion of the air stream penetrates from the direction according to arrow F in the interior of the ventilation duct, into the interior of the U profile 7, flows around the strip-shaped FA-substance carrier 6 there, from all sides, and exits from the U profile 7 again through narrow slits 21 in its crosspiece 9. In this manner, the FA-substance molecules formed in the interior of the U profile 7 by evaporation get into the air stream in the interior of the ventilation duct. There, the FA-substance molecules are picked up by the air stream and transported into the dissemination zone, while avoiding droplet contamination.

To promote flow in the interior of the U profile 7, an outside profile 22 is furthermore provided, which is composed of a central U-profile part 23 and lateral profile projections 24, which describe a lateral arch, in order to deflect the inflowing air in a direction onto the top of the rod-shaped hollow-profile carrier 25, in accordance with the curvature, so that flushing of the interior of the U profile 7 is promoted by means of generation of a partial vacuum. The arched profile projections 24 of the outer profile 22 are found on both sides of the central U-profile part 23, so that flow conditions that are approximately the same prevail in the event of a reversal of the through-flow direction (cf. Arrow f). To improve the heat insulation in the interior of the U profile 7, the profile cross-section shown in FIG. 3 also possesses a U profile 26 made of a heat-resistant plastic, which serves for heat insulation. In this manner, and by means of the tight encapsulation of the circulating FA-substance carrier 6 and of the heating rod 16 disposed between its upper run 14 and its lower run 15, in the interior of the U profile 7, it is possible to minimize the energy requirement for operation of the heating rod 16.

Installation of the evaporation apparatus is significantly simplified in that in place of a closed hollow profile, a hollow-profile carrier 25 composed of profiles 7, 18, 26, 23 composed of multiple parts is selected. In this way, the installations in the interior of the open U profile 7 can first be undertaken, and afterward, the U profile 7, with the installations, can be connected with the remaining profile parts 18, 26, 23, in simple manner.

Going beyond the components shown in FIG. 3, it is furthermore necessary to install the second deflection roller 27 in the inner end region of the rod-shaped hollow-profile carrier 25, and this can also take place essentially without hindrance, with an open U profile 7.

With the exception of the heat-insulating U profile 26, it is practical that all the other profiles can be configured as aluminum profiles. Preferably, a braided material of thin metal threads is possible as a strip material for the circulating FA-substance carrier 6, which offers the advantage that it possesses great capacity for taking up liquid FA substance, so that during application, no liquid level of any kind builds up; instead the liquid, which is applied to the metal strip in metered manner, is drawn into the braid cavities of the metal strip by capillary action, without delay. The metal strip furthermore has the advantage that it can be cleaned after extended use, without great effort.

Vertical installation positions are also possible because of the particularly advantageous capillary action of the metal strip, which is composed of bound metal threads, without any risk of dripping. The maintenance effort for the apparatus parts disposed in the interior of the ventilation duct is therefore low. The same holds true for the apparatus parts installed on the outside of the ventilation duct, which parts are unaffected by disadvantageous effects caused by air flow, in any case, and therefore guarantee good accessibility for maintenance purposes.

What is claimed is:

1. Method for molecular evaporation of fragrance substances and/or active substances (FA substances) and their dissemination by way of air streams in the surrounding air, in closed rooms, in ducts of ventilation and air conditioning systems, or the like, wherein FA substance, before its dissemination, is applied, in liquid form, to a strip-shaped FA-substance carrier (6), wherein the FA-substance carrier charged with FA substance has an air stream flowing against it transverse to its longitudinal axis,
characterized in that
the strip-shaped FA-substance carrier (6) is moved to circulate at least partly within a dissemination zone for FA substance, and that the FA substance to be evaporated is heated, directly or indirectly, to a temperature that promotes molecular evaporation, during its circulation on the FA-substance carrier (6).

2. Method according to claim 1,
characterized in that
the FA-substance carrier (6) is moved to circulate on rollers.

3. Method according to claim 1,
characterized in that
the FA-substance carrier (6) is moved at a variably adjustable speed.

4. Method according to claim 1,
characterized in that
the amount of the FA substance that is applied to the FA-substance carrier (6) is adjustable.

5. Method according to claim 4,
characterized in that
the FA substance is constantly renewed, during circulation of the FA-substance carrier (6), by means of metering in fresh FA substance.

6. Method according to claim 1,
characterized in that
the evaporation temperature amounts to between 40 and 100° C.

7. Apparatus for molecular evaporation and dissemination of fragrance substances/active substances by way of air streams in the surrounding air, in closed rooms, in ducts of ventilation and air conditioning systems, or the like, having a metering apparatus for application of FA substances, in liquid form, to a strip-shaped FA-substance carrier (6), which has an air stream flowing against it transverse to its longitudinal axis, at least partly within a dissemination zone, and wherein the metering apparatus is disposed outside of the dissemination zone,
characterized in that
the strip-shaped FA-substance carrier (6) can be moved on a support profile to circulate on rollers, and that a heating apparatus is provided, which essentially extends along the movement path of the FA-substance carrier (6).

8. Apparatus according to claim 7,
characterized in that
the strip-shaped FA-substance carrier (6) and the related heating apparatus are disposed encapsulated in the interior of a hollow-profile carrier (25), at least within the dissemination zone, except for incoming and outgoing ventilation openings.

9. Apparatus according to claim 7,
characterized in that
the FA-substance carrier (6) is configured as a strip that circulates between two deflection rollers (5, 27), and that the heating apparatus is disposed between an upper (14) and a lower run (15) of the circulating strip, in the form of an elongated heating rod (16).

10. Apparatus according to claim 9,
characterized in that
a deflection roller (5) is configured outside of the dissemination zone, as a drive roller, and driven by an electric motor (4) coupled with it.

11. Apparatus according to claim 7,
characterized in that
the FA-substance carrier (6) consists, as a circulating strip, of closely bound metal threads having a great capillary effect.

12. Apparatus according to claim 8,
characterized in that
the hollow-profile carrier (25) has narrow slit openings (20, 21) on opposite sides.

13. Apparatus according to claim 12,
characterized in that
the hollow-profile carrier (25) is composed of profile parts (7, 18).

14. Apparatus according to claim 13,
characterized in that
one or more profile parts (7) have slit-shaped openings (21).

15. Apparatus according to claim 13,
characterized in that
the profile parts (7, 18) are accommodated in a U-shaped outer profile (23) that has flow-advantageous profile projections (24) on one or both sides.

16. Apparatus according to claim 15,
characterized in that
a U profile (26) composed of heat-insulating material is inserted between the profile parts (7, 18) and the outer profile (23).

17. Apparatus for generating a door air curtain, with a blowing stream approximately parallel to a door opening, which stream exits from a flow duct for incoming air, through a grid that extends along a section of the door frame, characterized by an apparatus for evaporation and dissemination of fragrance substances and/or active substances according to claim 7, disposed entirely or partly outside of or within the flow duct, against which apparatus the incoming air flows transverse to its longitudinal axis.
